(19) United States
(12) Patent Application Publication
(10) Pub. No.: US 2024/0362658 A1
(43) Pub. Date: Oct. 31, 2024

(54) REWARD GRANTING SYSTEM, SERVER SYSTEM, REWARD GRANTING METHOD AND PROGRAM

(71) Applicant: RAKUTEN GROUP, INC., Tokyo (JP)

(72) Inventors: Shiori NAGASE, Tokyo (JP); Kazuyuki Sato, Tokyo (JP)

(73) Assignee: RAKUTEN GROUP, INC., Tokyo (JP)

(21) Appl. No.: 18/029,047

(22) PCT Filed: Oct. 28, 2022
(86) PCT No.: PCT/JP2022/040543

§ 371 Date: Mar. 28, 2023

(51) Int. Cl. G06Q 30/0207; G06F 1/16; G16H 40/60

(52) U.S. Cl. CPC G06Q 30/0207; G06F 1/163; G16H 40/60

(57) ABSTRACT

At least one processor determines, based on reward information, whether a content of a reward satisfies a predetermined condition; at least one processor makes a first determination of, when the content of the reward does not satisfy the predetermined condition, whether user identification information is associated with terminal identification information on any one of a plurality of wearable terminals belonging to an operator; and at least one processor makes a second determination of whether the terminal identification information transmitted by a user terminal matches the terminal identification information on any one of the plurality of wearable terminals belonging to the operator.

### TECHNICAL FIELD

The present invention relates to a reward granting system, a server system, a reward granting method, and a program.

### BACKGROUND ART

There has been known a technology for measuring health data (for example, step count and heart rate) of a user by using a wearable terminal such as a watch-type terminal worn by the user, and managing the health of the user based on the measured health data by using, for example, a personal computer or a smartphone (see Patent Literature 1).

### CITATION LIST

### Patent Literature

[PTL1] JP 6507117 B2

### Non-Patent Literature

[NPL1] vitality.sumitomolife.co.jp/guide/partner/apple/

### SUMMARY OF INVENTION

### Technical Issue

In Non-Patent Literature 1, there is described a service in which health data of a user measured by a wearable terminal is converted into a reward such as points, and the reward is granted to the user within an app. Such a service can be expected to promote the use of the app as well as promote sales of the wearable terminal by operators providing the service.

Incidentally, when a reward is granted to a user, from the perspective of fraud prevention, it is preferred to introduce means for determining whether or not the wearable terminal of the user is associated with the operator providing the service. Without such means, for example, a reward may be granted even for health data measured by a wearable terminal purchased from an operator other than the service provider.

Ideally, as such means, it is preferred to introduce means for determining whether or not terminal identification information on the wearable terminal of the user matches the terminal identification information on any one of a plurality of wearable terminals managed by the operator.

However, when a determination as described above is performed in every case, there is an issue in that user convenience deteriorates. For security reasons, it is generally difficult for an operator to acquire the terminal identification information on the wearable terminal of the user. Thus, in the case of performing a determination as described above, the user is required to acquire the terminal identification information on the wearable terminal of the user and input the terminal identification information to an app, and hence user convenience deteriorates.

The present invention has been made in view of the above-mentioned issue, and it is an object thereof to provide a reward granting system, a reward granting method, and a program which are convenient to a user.

### Solution to Issue

(1) According to one embodiment of the present invention, there is provided a reward granting system including: a user terminal; and a server system of an operator that provides a service to a user, the user terminal including: acquisition means for acquiring health data measured by a wearable terminal of the user; determination means for determining, based on the health data, a reward to be granted to the user; and first transmission means for transmitting, to the server system, reward information indicating a content of the reward and user identification information on the user, the server system including: determination means for determining, based on the reward information, whether the content of the reward satisfies a predetermined condition; first determination means for determining, when the content of the reward does not satisfy the predetermined condition, whether the user identification information is associated with terminal identification information on any one of a plurality of wearable terminals belonging to the operator; and request means for transmitting, when the content of the reward satisfies the predetermined condition, an input request of the terminal identification information on the wearable terminal of the user to the user terminal, the user terminal further including second transmission means for transmitting, to the server system, the terminal identification information input by the user in response to the request, the server system further including: second determination means for determining whether the terminal identification information transmitted by the user terminal matches the terminal identification information on any one of the plurality of wearable terminals belonging to the operator; and registration means for registering the reward information and the user identification information in association with each other based on a result of the determination made by the first determination means or a result of the determination made by the second determination means.

(2) In the reward granting system according to Item (1), the server system may further include first storage means for storing the terminal identification information on any one of the plurality of wearable terminals belonging to the operator in association with the user identification information.

(3) In the reward granting system according to Item (1) or (2), the server system may further include second storage means for storing the terminal identification information on each of the plurality of wearable terminals belonging to the operator.

(4) In the reward granting system according to any one of Items (1) to (3), the first determination means may be configured to further determine whether the user possesses any one of the plurality of wearable terminals belonging to the operator.

(5) In the reward granting system according to Item (4), the server system may further include third storage means for storing, in association the with user identification information, possession information indicating whether the user possesses any one of the plurality of wearable terminals belonging to the operator.

(6) In the reward granting system according to any one of Items (1) to (5), the service may include a service relating to use of a communication line, and the second determination means may be configured to further determine whether the communication line is usable via the wearable terminal of the user.

(7) In the reward granting system according to Item (6), the server system may further include fourth storage means for storing, in association with the user identification information or the terminal identification information, contract information indicating whether the communication line is usable via the wearable terminal of the user.

(8) According to one embodiment of the present invention, there is provided a server system of an operator that provides a service to a user, the server system being configured to communicate to and from a user terminal, the server system including: first reception means for receiving, from the user terminal, reward information indicating a content of a reward to be granted to the user and user identification information on the user, the reward being determined based on health data measured by a wearable terminal of the user; determination means for determining, based on the reward information, whether the content of the reward satisfies a predetermined condition; first determination means for determining, when the content of the reward does not satisfy the predetermined condition, whether the user identification information is associated with terminal identification information on any one of a plurality of wearable terminals belonging to the operator; request means for transmitting, when the content of the reward satisfies the predetermined condition, an input request of the terminal identification information on the wearable terminal of the user to the user terminal; second reception means for receiving, from the user terminal, the terminal identification information input by the user in response to the request; second determination means for determining whether the terminal identification information received from the user terminal matches the terminal identification information on any one of the plurality of wearable terminals belonging to the operator; and registration means for registering the reward information and the user identification information in association with each other based on a result of the determination made by the first determination means or a result of the determination made by the second determination means.

(9) According to one embodiment of the present invention, there is provided a reward granting method to be executed by a user terminal and a server system of an operator that provides a service to a user, the reward granting method including: executing, by the user terminal: an acquisition step of acquiring health data measured by a wearable terminal of the user; a determination step of determining, based on the health data, a reward to be granted to the user; and a first transmission step of transmitting, to the server system, reward information indicating a content of the reward and user identification information on the user; executing, by the server system: a determination step of determining, based on the reward information, whether the content of the reward satisfies a predetermined condition; a first determination step of determining, when the content of the reward does not satisfy the predetermined condition, whether the user identification information is associated with terminal identification information on any one of a plurality of wearable terminals belonging to the operator; and a request step of transmitting, satisfies the predetermined when the content of the reward condition, an input request of the terminal identification information on the wearable terminal of the user to the user terminal; further executing, by the user terminal, a second transmission step of transmitting, to the server system, the terminal identification information input by the user in response to the request; and further executing, by the server system: a second determination step of determining whether the terminal identification information transmitted from the user terminal matches the terminal identification information on any one of the plurality of wearable terminals belonging to the operator; and a registration step of registering the reward information and the user identification information in association with each other based on a result of the determination made in the first determination step or a result of the determination made in the second determination step.

(10) According to one embodiment of the present invention, there is provided a program for causing a server system of an operator that provides a service to a user, the server system being configured to communicate to and from a user terminal, to function as: first reception means for receiving, from the user terminal, reward information indicating a content of a reward to be granted to the user and user identification information on the user, the reward being determined based on health data measured by a wearable terminal of the user; determination means for determining, based on the reward information, whether the content of the reward satisfies a predetermined condition; first determination means for determining, when the content of the reward does not satisfy the predetermined condition, whether the user identification information is associated with terminal identification information on any one of a plurality of wearable terminals belonging to the operator; request means for transmitting, when the content of the reward satisfies the predetermined condition, an input request of the terminal identification information on the wearable terminal of the user to the user terminal; second reception means for receiving, from the user terminal, the terminal identification information input by the user in response to the request; second determination means for determining whether the terminal identification information received from the user terminal matches the terminal identification information on any one of the plurality of wearable terminals belonging to the operator; and registration means for registering the reward information and the user identification information in association with each other based on a result of the determination made by the first determination means or a result of the determination made by the second determination means.

### BRIEF DESCRIPTION OF DRAWINGS

FIG. 1 is a diagram for illustrating a hardware configuration of a reward granting system according to an embodiment of the present invention.

FIG. 2 is a diagram for illustrating an outline of the reward granting system according to the embodiment of the present invention.

FIG. 3 is a diagram for illustrating an outline of the reward granting system according to the embodiment of the present invention.

FIG. 4 is a functional block diagram for illustrating an example of functions implemented by the reward granting system according to the embodiment of the present invention.

FIG. 5 is a table for showing a data storage example of a reward information database.

FIG. 6 is a table for showing a data storage example of a user database.

FIG. 7 is a flow chart for illustrating an example of a process executed by the reward granting system according to the embodiment of the present invention.

FIG. 8 is a flow chart for illustrating an example of a process executed by the reward granting system according to the embodiment of the present invention.

### DESCRIPTION OF EMBODIMENTS

### [1. Hardware Configuration of Reward Granting System]

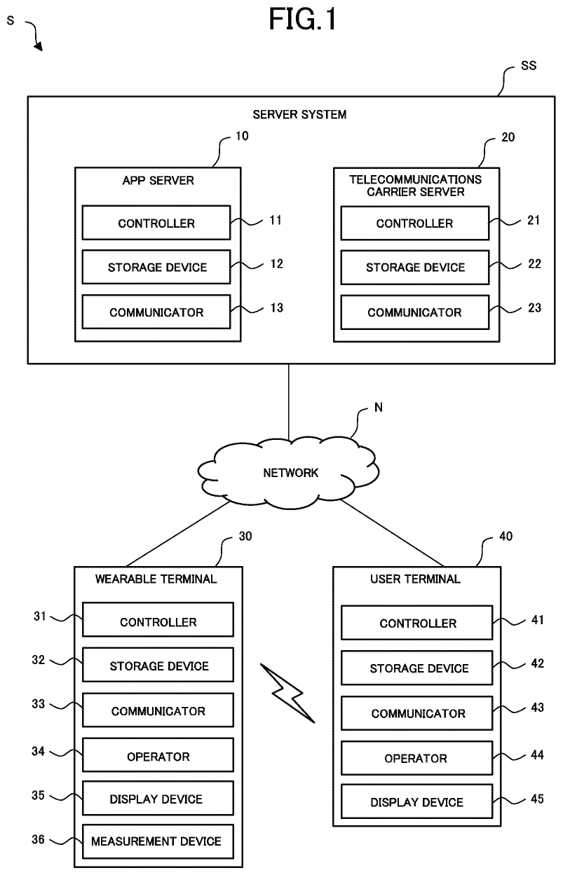

A reward granting system according to an embodiment of the present invention is now described. FIG. 1 is a diagram for illustrating a hardware configuration of the reward granting system according to the embodiment of the present invention. As illustrated in FIG. 1, a reward granting system S includes a server system SS, a wearable terminal 30, and a user terminal 40. The server system SS includes an app server 10 and a telecommunications carrier server 20. Each of the app server 10, the telecommunications carrier server 20, the wearable terminal 30, and the user terminal 40 can be connected to a network N such as the Internet. In FIG. 1, one app server 10, one telecommunications carrier server 20, one wearable terminal 30, and one user terminal 40 are illustrated, but there may be a plurality of those. Further, it is not required that the app server 10 and the telecommunications carrier server 20 be separate, and the functions of the app server 10 and the functions of the telecommunications carrier server 20 may be implemented by one server computer.

The app server 10 is a server computer which provides various services to users through application software (hereinafter simply referred to as “app”) executed by the user terminal 40. The app server 10 corresponds to an operator which provides a service to a user through an app. In the present embodiment, there is described a case in which an insurance service is provided to a user through an app, that is, a case in which the operator is an operator which provides an insurance service (hereinafter referred to as “insurance carrier”). As a matter of course, the service is not limited to the example of this embodiment. As described in detail later, the insurance carrier provides a service in which a reward corresponding to health data, such as a step count, measured by the wearable terminal 30 of the user is granted to the user in the provision of the insurance service.

The app server 10 includes a controller 11, a storage device 12, and a communicator 13. The controller 11 includes at least one processor. The storage device 12 includes a volatile memory such as a RAM and a nonvolatile memory such as a hard disk drive. The communicator 13 includes at least one of a communication interface for wired communication or a communication interface for wireless communication.

The telecommunications carrier server 20 is a server computer which provides a service relating to the use of a communication line. The telecommunications carrier server 20 corresponds to a telecommunications carrier which provides users with services relating to the use of communication lines (hereinafter referred to as “telecommunications carrier”). In the present embodiment, the user is a user of the insurance carrier as well as a user of the telecommunications carrier. Further, in the present embodiment, the insurance carrier and the telecommunications carrier are different operators, but the insurance carrier and the telecommunications carrier cooperate with each other. For example, the insurance carrier and the telecommunications carrier may each be a subsidiary of the same group company. Further, the insurance carrier and the telecommunications carrier may be the same operator. In that case, as described above, the functions of the app server 10 and the functions of the telecommunications carrier server 20 may be implemented by one server computer. The telecommunications carrier server 20 includes a controller 21, a storage device 22, and a communicator 23. The physical configurations of the controller 21, the storage device 22, and the communicator 23 are the same as the physical configurations of the controller 11, the storage device 12, and the communicator 13, respectively.

The wearable terminal 30 is a computer such as a watch-type computer worn by the user. The wearable terminal 30 includes a controller 31, a storage device 32, a communicator 33, an operator 34, a display device 35, and a measurement device 36. The physical configurations of the controller 31, the storage device 32, and the communicator 33 are the same as the physical configurations of the controller 11, the storage device 12, and the communicator 13, respectively. The operator 34 is an input device such as a touch panel. The display device 35 is a liquid crystal display or an organic EL display. The measurement device 36 includes an acceleration sensor configured to measure the step count of the user and a heart rate sensor configured to measure the heart rate of the user.

The user terminal 40 is a computer to be operated by the user. For example, the user terminal 30 is a smartphone, a tablet terminal, or a personal computer. The user terminal 30 includes a controller 41, a storage device 42, a communicator 43, an operator 44, and a display device 45. The physical configurations of the controller 41, the storage device 42, the communicator 43, the operator 44, and the display device 45 are the same as the physical configurations of the controller 31, the storage device 32, the communicator 33, the operator 34, and the display device 35, respectively.

At least one of the programs or data stored in the storage devices 12, 22, 32, and 42 may be supplied via the network N. In addition, each of the app server 10, the telecommunications carrier server 20, the user terminal 30, and the wearable terminal 40 may include at least one of a reader (for example, an optical disc drive or a memory card slot) configured to read a computer-readable information storage medium or an input/output device (for example, a USB port) configured to input and output of data to and from an external device. For example, at least one of the programs or data stored in the information storage medium may be supplied via at least one of the reader or the input/output device.

### [2. Outline of Reward Granting System]

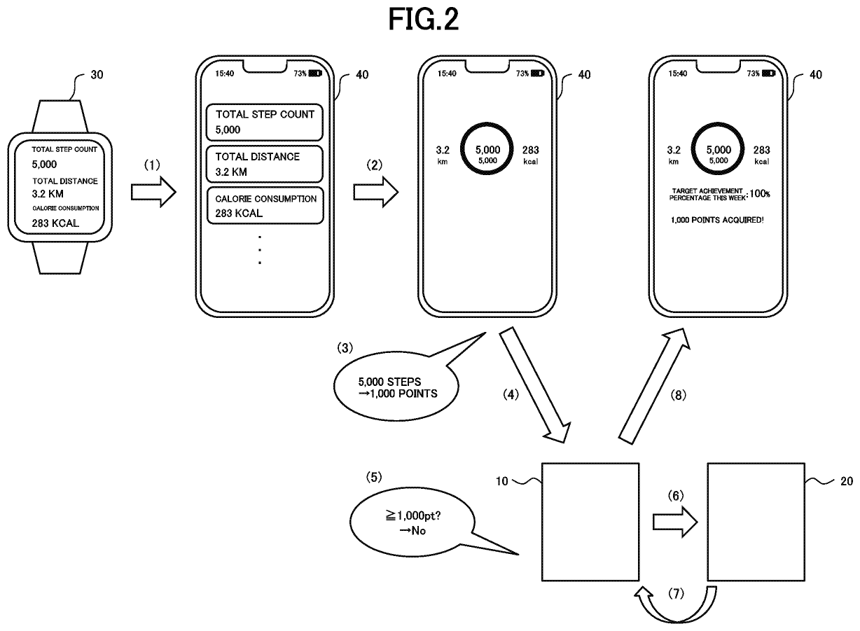

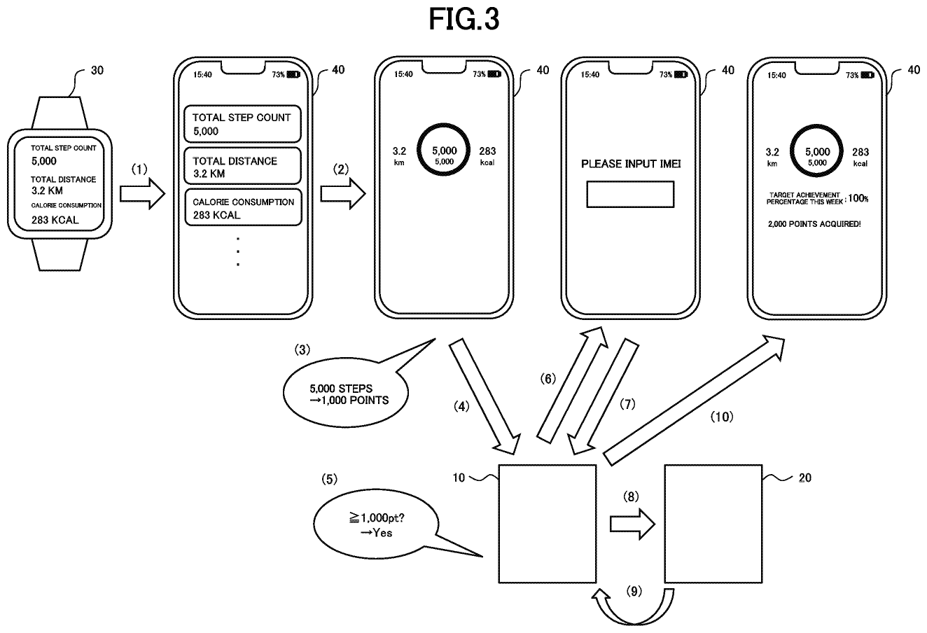

An outline of the reward granting system S of this embodiment is now described with reference to FIG. 2 and FIG. 3. FIG. 2 and FIG. 3 are diagrams for illustrating an outline of the reward granting system according to the embodiment of the present invention.

First, the wearable terminal 30 worn by the user measures the health data of the user. The measured health data is transmitted to the user terminal 40 by publicly-known communication means, such as Wi-Fi (trademark) and Bluetooth (trademark) ((1) of FIG. 2). A default healthcare app is installed in the user terminal 40, and the health data is managed by the healthcare app.

The health data managed by the default health care app is shared with the app associated with the app server 10 via an API ((2) of FIG. 2). Then, the step count in the health data is converted into points by the app associated with the app server 10 ((3) of FIG. 2). The converted point information is transmitted from the user terminal 40 to the app server 10 together with a user ID of the user ((4) of FIG. 2).

The app server 10 determines whether or not the points are equal to or more than a predetermined value based on the transmitted point information ((5) of FIG. 2). When the points are less than the predetermined value, the user ID is transmitted to the telecommunications carrier server 20 ((6) of FIG. 2). The telecommunications carrier server 20 determines, based on the transmitted user ID, whether or not the telecommunications carrier has sold a wearable device to the user. When it is determined that the telecommunications carrier has sold a wearable device to the user, the telecommunications carrier server 20 transmits, to the app server 10, a notification to the effect that points can be granted to the user ((7) of FIG. 2). The app server 10 registers the user ID and information on the points to be granted in association with each other, and displays a screen indicating that the points have been granted to the user on the display device 45 of the user terminal 40 ((8) of FIG. 2).

Meanwhile, when the points are equal to or more than the predetermined value, the app server 10 transmits an input request of terminal identification information on the wearable terminal 30 of the user to the user terminal 40 ((6) of FIG. 3). In the present embodiment, the terminal identification information is a terminal identification number, referred to as “International Mobile Equipment Identity (IMEI),” assigned to each terminal, but the terminal identification information is not limited to the example. When the user terminal 40 receives the request, the user terminal 40 displays a screen instructing the user to input the IMEI. In response to this, the user inputs the IMEI, and when the input IMEI is transmitted to the app server 10 ((7) of FIG. 3), the app server 30 transmits the IMEI to the telecommunications carrier server 20 ((8) of FIG. 3).

The telecommunications carrier server 20 determines, based on the transmitted IMEI, whether or not the wearable device of the user has been purchased from the telecommunications carrier. When it is determined that the wearable device of the user has been purchased from the telecommunications carrier, the telecommunications carrier server 20 transmits to the app server 10 a notification to the effect that the points can be granted to the user ((9) of FIG. 3). The app server 10 registers the user ID and the information on the points to be granted in association with each other, and displays a screen indicating that the points have been granted to the user on the display device 45 of the user terminal 40 ((10) of FIG. 3).

As described above, the reward granting system S according to the embodiment of the present invention uses different fraud prevention means depending on the amount of points to be granted to the user. From the standpoint of fraud prevention, it is desirable to determine, in all cases, whether or not the wearable device of the user has been purchased from the telecommunications carrier, specifically, it is desirable to determine whether or not the IMEI of the wearable device of the user matches the IMEI one the wearable devices managed by the of any of telecommunications carrier. However, when such fraud prevention means is adopted, the user is constantly forced to perform an operation to input the IMEI, and user convenience deteriorates. In this respect, according to the reward granting system S of the embodiment of the present invention, the user is required to input the IMEI only when the points to be granted are more than a predetermined value, and thus user convenience can be improved while preventing fraud. The details of the technology are now described.

### [3. Functions Implemented by Reward Granting System]

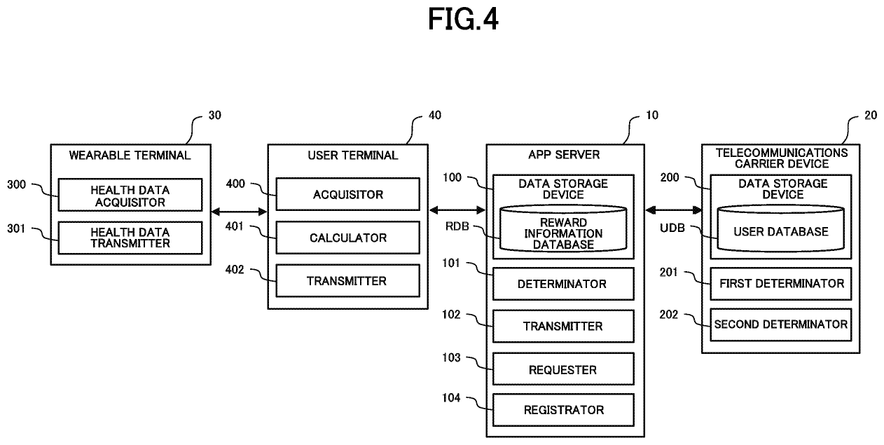

The functions implemented by the reward granting system S of this embodiment are now described with reference to FIG. 4. FIG. 4 is a functional block diagram for illustrating an example of functions implemented by the reward granting system according to the embodiment of the present invention.

### [3-1. Functions Implemented by Wearable Terminal]

As illustrated in FIG. 4, a health data acquisitor 300 and a health data transmitter 301 are implemented by the wearable terminal 30. The health data acquisitor 300 is implemented mainly by the controller 31 and the measurement device 36. The health data transmitter 301 is implemented mainly by the controller 31 and the communicator 33. The health data acquisitor 300 acquires health data of the user. The health data includes various types of health data, for example, a step count, a walking distance, and a heart rate. The health data is transmitted to the user terminal 40 by the health data transmitter 301. In the present embodiment, the health data transmitter 301 automatically transmits the health data to the user terminal 40, but the health data transmitter 301 may transmit the health data to the user terminal 40 in response to an operation by the user.

### [3-2. Functions Implemented by User Terminal]

As illustrated in FIG. 4, an acquisitor 400, a calculator 401, and a transmitter 402 are implemented by the user terminal 40. The acquisitor 400 is implemented mainly by the controller 41 and the communicator 43. The calculator 401 is implemented mainly by the controller 41. The transmitter 402 is implemented mainly by the controller 41 and the communicator 43.

### [Acquisitor]

The acquisitor 400 acquires the health data measured by the wearable terminal 30 of the user. In the present embodiment, the health data transmitted by the health data transmitter 301 of the wearable terminal 30 is temporarily stored in the storage device 42 of the user terminal 40. Thus, the acquisitor 400 acquires the health data from the storage device 42. However, the acquisitor 400 may acquire the health data directly from the wearable terminal 30. Moreover, the health data transmitter 301 of the wearable terminal 30 may transmit the health data to an external information storage medium or computer other than the user terminal 40. In this case, the acquisitor 400 may acquire the health data from the external information storage medium or computer.

### [Calculator]

The calculator 401 calculates (determines) points as a reward to be granted to the user based on the health data. In the present embodiment, the calculator 401 calculates the points to be granted to the user based on step count data out of the health data, but the calculator 401 may calculate the points based on health data other than the step count data (for example, heart rate data). As other examples, the calculator 401 may calculate the points based on all the data elements in the health data, or may calculate the points based on a combination of two or more data elements in the health data. Further, in the present embodiment, a case in which the reward granted to the user is points that can be used at an insurance carrier is described, but the reward granted to the user is not limited to this example. The reward may be, for example, a lottery ticket voucher, a merchandise voucher, or electronic money.

Incidentally, in the related art, a point calculation process as described above is executed by a server. However, when the point calculation process is executed by a server, there is an issue in that it is not easy to change or add process content for security reasons. In this regard, in the present embodiment, because the calculator 401 of the user terminal 40 executes the point calculation process, it is easier to change or add process content. In a case in which consideration is not given to changes or additions to the process content, the app server 10 or the telecommunications carrier server 20 may include a function corresponding to the calculator 401.

### [Transmitter of User Terminal]

The transmitter 402 of the user terminal 40 transmits the point information (reward information indicating the content of the reward) and the user ID, which is user identification information on the user, to the app server 10. In the present embodiment, the transmitter 402 transmits the point information as the reward information, but when the reward is a lottery ticket voucher, the reward information may be the number of lottery ticket vouchers. Further, when the reward is a merchandise voucher, the reward information may be the content of the merchandise that is to be exchanged with the merchandise voucher. When the reward is electronic money, the reward information may be the amount of electronic money. The user identification information is information that can identify a user of the insurance carrier. Specifically, the user ID is a user identification number at the insurance carrier. The user

ID may be common between the insurance carrier and the telecommunications carrier. Further, the user identification information is not limited to the user ID, and may be a user account name or an electronic mail address, for example.

In the present embodiment, the transmitter 402 further transmits an International Mobile Scriber Identity (IMSI) to the app server 10. In the present embodiment, the insurance carrier and the telecommunications carrier are different operators. The above-mentioned user ID functions as the user identification information for the insurance carrier, and the IMSI functions as the user identification information for the telecommunications carrier. That is, in the present embodiment, the user ID is used as first user identification information, and the IMSI is used as second user identification information, thereby increasing the security strength. The second user identification information may be any information as long as the second user identification information functions as user identification information for the telecommunications carrier.

The transmitter 402 may indirectly transmit the point information, the user ID, and the IMSI to the app server 10. Specifically, the transmitter 402 may transmit the point information, the user ID, and the IMSI to an external server or the like, and then the external server or the like may transmit those pieces of information to the app server 10. The same applies to a transmitter 102 and a requestor 103 of the app server 10, which are described later.

Further, the transmitter 402 transmits the IMEI, which is terminal identification information input by the user, to the app server 10 in response to a request transmitted from the requestor 102 of the app server 10, which is described later. The terminal identification information is information that can identify the wearable terminal 30 of the user. The IMEI is a terminal identification number assigned to each wearable terminal, as described above. When the input request the IMEI is received from the requestor 103, the user terminal 40 displays a screen instructing the user to input the IMEI on the display device 45. The user can input the IMEI to the user terminal 40 by copying and pasting the IMEI from a device information screen of the user terminal 40, for example.

### [3-3. Functions Implemented by App Server]

As illustrated in FIG. 4, a data storage device 100, a determinator 101, the transmitter 102, the requestor 103, and a registrator 104 are implemented by the app server 10. The data storage device 100 is implemented mainly by the storage device 12. The determinator 101 is implemented mainly by the controller 31. The transmitter 102 and the requestor 103 are implemented mainly by the controller 31 and the communicator 33. The registrator 104 is implemented mainly by the controller 31 and the storage device 32.

### [Data Storage Device of App Server]

The data storage device 100 of the app server 10 stores a reward information database RDB. The details of the reward information database RDB are described later.

### [Determinator]

The determinator 101 determines, based on the point information transmitted by the transmitter 402 of the user terminal 40, whether or not the points are equal to or more than a predetermined value (whether or not the content of the reward satisfies a predetermined condition). The determinator 101 may also determine whether or not the points are more than the predetermined value. For example, when the reward is a lottery ticket voucher, the determinator 101 may determine whether or not the number of lottery ticket vouchers is equal to or more than a predetermined number. Further, when the reward is a merchandise voucher, the determinator 101 may determine whether or not the merchandise to be exchanged for the merchandise voucher corresponds to merchandise having a predetermined rank or higher. Specifically, there is considered a case in which, as the merchandise that can be exchanged for a merchandise voucher, the first-ranked merchandise is a hand massager, the second-ranked merchandise is coupon tickets for a deluxe public bath, and the third-ranked merchandise is one case of canned juice. In this case, it may be determined whether or not the merchandise to be exchanged for the merchandise voucher is, for example, the second rank or higher. Moreover, when the reward is electronic money, the determinator 101 may determine whether or not the amount of electronic money is equal to or more than a predetermined amount. In short, it is only required that the determinator 101 determine whether or not the content of the reward satisfies a predetermined condition.

### [Transmitter of App Server]

The transmitter 102 of the app server 10 transmits the user ID and the IMSI to the telecommunications carrier server 20 when the points are less than the predetermined value.

In addition, the transmitter 102 transmits, to the telecommunications carrier server 20, the IMEI input to the user terminal 40 by the user in response to a request from the requestor 103, which is described later, and transmitted to the app server 10.

### [Requestor]

The requestor 103 transmits, when the points are equal to or more than the predetermined value, an input request of the IMEI of the wearable terminal 30 of the user to the user terminal 40. The input of the IMEI to the user terminal 40 by the user is as described above.

### [Registrator]

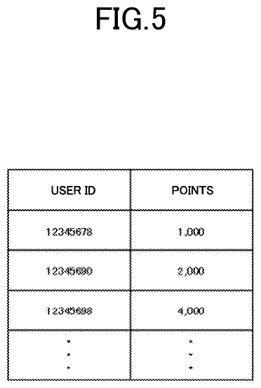

The registrator 105 registers the point information and the user ID in association with each other based on determination result of a first determinator 201 or a determination result of a second determinator 202 of the telecommunications carrier server 20, which are described later. Specifically, based on a determination result of the first determinator 201 that the user ID is associated with the IMEI of any one of the plurality of wearable terminals belonging to the telecommunications carrier, the registrator 105 stores the point information and the user ID in the data storage device 100 in association with each other. As another example, based on a determination result of the second determinator 201 that the IMEI transmitted by the user terminal 40 matches the IMEI of any one of the plurality of wearable terminals belonging to the telecommunications carrier, the registrator 105 stores the point information and the user ID in the data storage device 100 in association with each other. In this case, as shown in FIG. 5, the data in which the point information and the user ID are associated with each other is stored in the reward information database RDB of the data storage device 100. The app server 10 reads out the reward information database RDB as appropriate, and transmits a notification to the effect that points have been granted to the user to the user terminal 40. In response, as described with reference to FIG. 2 and FIG. 3, the user terminal 40 displays a screen indicating that the points have been granted to the user on the display device 45.

In the present embodiment, there has been described a case in which the reward information database RDB is stored in the app server 10, but the reward information database RDB may be stored in an external information storage medium or computer.

### [3-4. Functions Implemented by Telecommunications Carrier Server]

As illustrated in FIG. 4, a data storage device 200, the first determinator 201, and the second determinator 202 are implemented by the telecommunications carrier server. The data storage device 200 is implemented mainly by the storage device 32. The first determinator 201 and the second determinator 202 are implemented mainly by the controller 31.

### [Data Storage Device of Telecommunications Carrier Server]

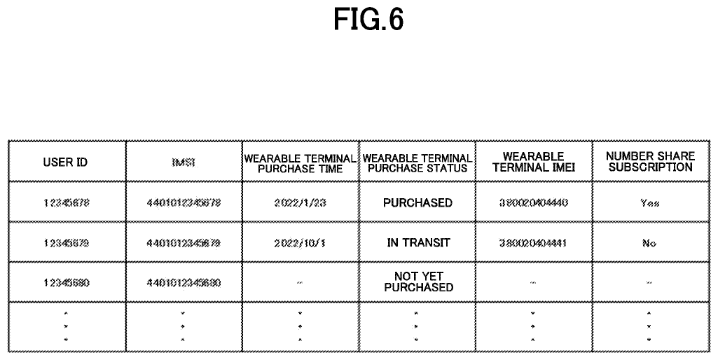

The data storage device 200 of the telecommunications carrier server 20 stores a user database UDB. Details of the user database UDB are now described with reference to FIG. 6. FIG. 6 is a table for showing a data storage example of the user database. As shown in FIG. 6, the user database UDB stores the user ID, the IMSI, a purchase time of the wearable terminal, a purchase status of the wearable terminal, the IMEI of the wearable terminal, and number sharing service subscription information in association with each other.

The telecommunications carrier server 20 stores the IMEI of each of a plurality of wearable terminals belonging to the telecommunications carrier based on the user database UDB. Further, as shown in FIG. 6, the telecommunications carrier server 20 stores, based on the user database UDB, the IMEI of any one of the plurality of wearable terminals belonging to the telecommunications carrier in association with each user ID. The plurality of wearable terminals belonging to the telecommunications carrier is, specifically, a plurality of wearable terminals sold by the telecommunications carrier. Moreover, as described later, when the telecommunications carrier leases out wearable terminals, the plurality of wearable terminals belonging to the telecommunications carrier may be the plurality of wearable terminals leased out by the telecommunications carrier.

Further, in the present embodiment, the telecommunications carrier server 20 stores a wearable terminal purchase status in association with each user ID based on the user database UDB. The wearable terminal purchase status is information indicating the wearable terminal purchase status of each user. Specifically, as shown in FIG. 6, the purchase status of each wearable terminal is indicated by expressions such as “purchased,” “in transit,” or “not yet purchased.” When the purchase status is stored as “purchased” for a certain user ID, this means that the user corresponding to that user ID possesses a wearable terminal. That is, the wearable terminal purchase status is possession information indicating whether or not the user possesses any one of the plurality of wearable terminals belonging to the telecommunications carrier.

As used herein, “possession” includes not only a state in which the wearable terminal is actually being worn, but also a state in which the wearable terminal is kept under the control of the user. Further, it does not matter whether the wearable terminal is owned by the user or by the telecommunications carrier. That is, in the present embodiment, the wearable terminal is sold to the user by the telecommunications carriers, but the wearable terminal may be leased out to the user by the telecommunications carrier.

Further, the telecommunications carrier server 20 stores number share service subscription information in association with the user IDs or the IMEIs based on the user database UDB. A number sharing service is a service in which the user terminal 40 and the wearable terminal 30 can share the phone number of the user for the communication line of the telecommunications carrier. That is, the number sharing service subscription information is contract information indicating whether or not a communication line can be used via the wearable terminal of the user. Specifically, as shown in FIG. 6, the number sharing service subscription information indicates whether or not the user has subscribed to the number sharing service by using an expression such as “Yes” or “No.” The contract information is not limited to number sharing service subscription information, and may be, for example, SIM registration information or a SIM line contract status.

In the present embodiment, a case in which the user database UDB is stored in the telecommunications carrier server 20 has been described, but the user database UDB may be stored in an external information storage medium or computer.

### [First Determinator]

The first determinator 201 determines, when the points transmitted from the app server 10 are less than the predetermined value, whether or not the user ID is associated with the IMEI of any one of the plurality of wearable terminals belonging to the telecommunications carrier. In addition, in the present embodiment, the first determinator 201 further determines whether or not the IMSI is associated with the IMEI of any one of the plurality of wearable terminals belonging to the telecommunications carrier.

Moreover, in the present embodiment, the first determinator 201 refers to the wearable terminal purchase status in the user database UDB to determine whether or not the wearable terminal purchase status stored in association with the user ID is “purchased” (whether or not the user possesses any one of the plurality of wearable terminals belonging to the telecommunications carrier). The first determinator 201 performs those determinations by referring to the user database UDB.

### [Second Determinator]

The second determinator 202 determines whether or not the IMEI transmitted by the user terminal 40 matches the IMEI of any one of the plurality of wearable terminals belonging to the telecommunications carrier. Further, in the present embodiment, the second determinator 202 determines whether or not the number sharing service subscription information stored in association with the user ID is “Yes” (whether or not the communication line can be used via the wearable terminal of the user). The second determinator 202 performs those determinations by referring to the user database UDB.

### [4. Process Executed by Reward Granting System]

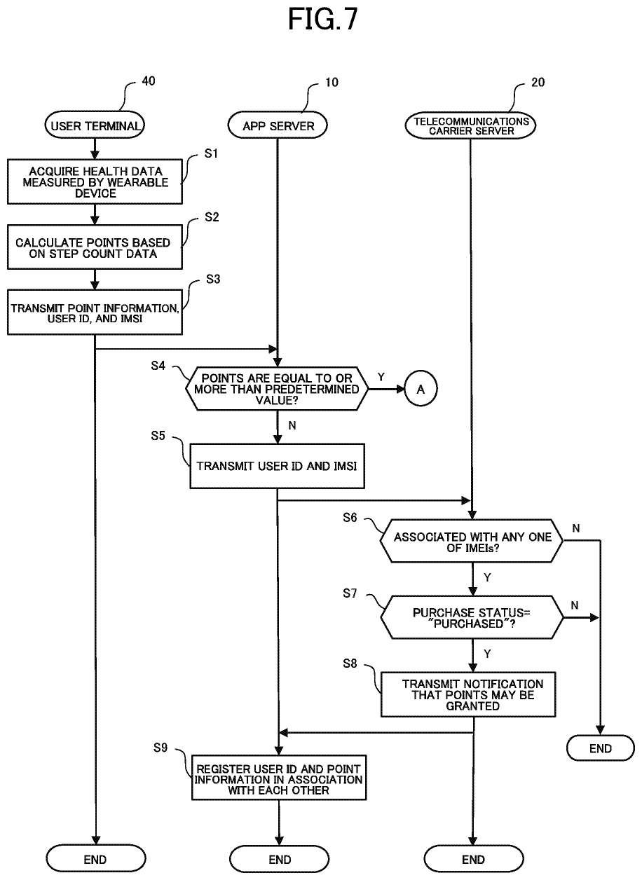

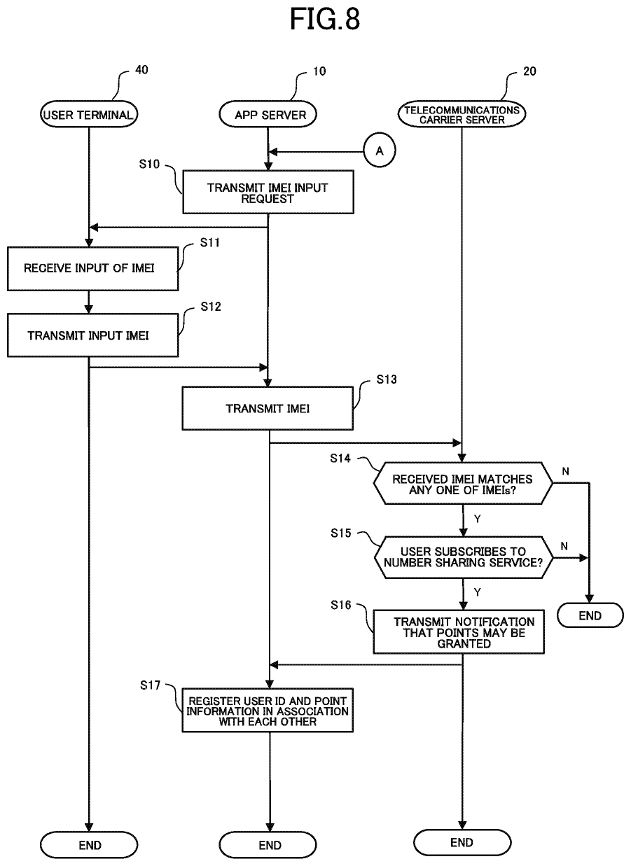

There is now described a process executed by the reward granting system S according to the embodiment of the present invention with reference to FIG. 7 and FIG. 8. FIG. 7 and FIG. 8 are flow charts for illustrating an example of the process executed by the reward granting system according to the embodiment of the present invention. The process illustrated in each of FIG. 7 and FIG. 8 is executed by the controllers 11, 21, and 41 operating in accordance with the programs stored in the storage devices 12, 22, and 42, respectively. The process of each of FIG. 7 and FIG. 8 is an example of a process executed by the functional blocks of FIG. 4.

As illustrated in FIG. 7, the user terminal 40 acquires the health data measured by the wearable terminal 30 of the user (Step S1). The user terminal 40 calculates the points to be granted to the user based on the step count in the acquired health data (Step S2), transmits the point information, the user ID, and the IMSI to the app server 10 (Step S3), and ends the process.

When the app server 10 receives the point information, the user ID, and the IMSI from the user terminal 40, the app server determines whether or not the points are equal to or more than a predetermined value (Step S4). When it is determined in Step S4 that the points are less than the predetermined value (“N” in Step S4), the app server 10 transmits the user ID and the IMSI to the telecommunications carrier server 20 (Step S5).

The telecommunications carrier server 20 receives the user ID and the IMSI from the app server 10, refers to the user database UDB, and determines whether or not the received user ID and IMSI are associated with the IMEI of any one of the plurality of wearable terminals belonging to the telecommunications carrier (Step S6). When it is determined in Step S6 that there is such an association (“Y” in Step S6), the telecommunications carrier server 20 determines whether or not the wearable terminal purchase status stored in association with the received user ID is “purchased” (Step S7). When it is determined in Step S7 that the wearable terminal purchase status is “purchased” (“Y” in Step S7), the telecommunications carrier server 20 transmits a notification to the effect that the points may be granted to the user to the app server 10 (Step S8). The app server 10 receives the notification, stores the user ID and the point information in the data storage device 100 in association with each other (Step S9), and ends the process. The telecommunications carrier server 20 ends the process after Step S8. Further, when it is determined in Step S6 that there is not an association (“N” in Step S6) or when it is determined in Step S7 that the wearable terminal purchase status is not “purchased” (“N” in Step S7), the telecommunications carrier server 20 also directly ends the process.

Returning to Step S4, when it is determined that the points are equal to or more than the predetermined value (“Y” in Step S4), the process proceeds to FIG. 8, and the app server 10 transmits an IMEI input request to the user terminal 40 (Step S10). When the user terminal 40 receives the request, the user terminal 40 receives input of the IMEI from the user (Step S11) and transmits the IMEI input by the user to the app server 10 (Step S12), as described with reference to FIG. 3. The app server 10 transmits the IMEI received from the user terminal 40 to the telecommunications carrier server 20 (Step S13).

The telecommunications carrier server 20 determines whether or not the IMEI received from the user terminal 40 matches the IMEI of any one of the plurality of wearable terminals belonging to the telecommunications carrier (Step S14). When it is determined in Step S14 that there is a match (“Y” in Step S14), the telecommunications carrier server 20 determines whether or not the number sharing service subscription information stored in association with the received user ID or IMEI is “Yes” (Step S15). When it is determined in Step S15 that the number sharing service subscription information is “Yes” (“Y” in Step S15), the telecommunications carrier server 20 transmits a notification to the effect that the points may be granted to the user to the app server 10 (Step S16). The app server 10 receives the notification, stores the user ID and the point information in the data storage device 100 in association with each other (Step S17), and ends the process. The telecommunications carrier server 20 ends the process after Step S16. Further, when it is determined in Step S14 that there is not a match (“N” in Step S6) or when it is determined in Step S7 that the number sharing service subscription information is “No” (“N” in Step S7), the telecommunications carrier server 20 also directly ends the process.

According to the reward granting system S of the above-mentioned embodiment, different fraud prevention means is used depending on whether or not the content of the reward to be granted to the user satisfies a predetermined condition. As a result, the user is not required to input the IMEI in all cases, and hence user convenience is excellent.

1. A reward granting system, comprising:
a user terminal; and
a server system of an operator that provides a service to a user,
the user terminal including at least one processor configured to:
acquire health data measured by a wearable terminal of the user;
determine, based on the health data, a reward to be granted to the user; and
transmit, to the server system, reward information indicating a content of the reward and user identification information on the user,
the server system including at least one processor configured to:
determine, based on the reward information, whether the content of the reward satisfies a predetermined condition;
make a first determination of, when the content of the reward does not satisfy the predetermined condition, whether the user identification information is associated with terminal identification information on any one of a plurality of wearable terminals belonging to the operator; and
transmit, when the content of the reward satisfies the predetermined condition, an input request of the terminal identification information on the wearable terminal of the user to the user terminal,
the at least one processor included in the user terminal is further configured to transmit, to the server system, the terminal identification information input by the user in response to the request,
the at least one processor included in the server system is further configured to:
make a second determination of whether the terminal identification information transmitted by the user terminal matches the terminal identification information on any one of the plurality of wearable terminals belonging to the operator; and
register the reward information and the user identification information in association with each other based on a result of the first determination or a result of the second determination.

2. The reward granting system according to claim 1, wherein the server system further includes a storage configured to store the terminal identification information on any one of the plurality of wearable terminals belonging to the operator in association with the user identification information.

3. The reward granting system according to claim 1, wherein the server system further includes a storage configured to store the terminal identification information on each of the plurality of wearable terminals belonging to the operator.

4. The reward granting system according to claim 1, the at least one processor included in the server system is configured to, when the content of the reward does not satisfy the predetermined condition, further determine whether the user possesses any one of the plurality of wearable terminals belonging to the operator.

5. The reward granting system according to claim 4, wherein the server system further includes a storage configured to store, in association with the user identification information, possession information indicating whether the user possesses any one of the plurality of wearable terminals belonging to the operator.

6. The reward granting system according to claim 1,
wherein the service includes a service relating to use of a communication line, and
wherein the at least one processor included in the server system, when the server system receives the terminal identification information input by the user in response to the request, is configured to further determine whether the communication line is usable via the wearable terminal of the user.

7. The reward granting system according to claim 6, wherein the server system further includes a storage configured to store, in association with the user identification information or the terminal identification information, contract information indicating whether the communication line is usable via the wearable terminal of the user.

8. A server system of an operator that provides a service to a user, the server system being configured to communicate to and from a user terminal, the server system comprising at least one processor configured to:
receive, from the user terminal, reward information indicating a content of a reward to be granted to the user and user identification information on the user, the reward being determined based on health data measured by a wearable terminal of the user;
determine, based on the reward information, whether the content of the reward satisfies a predetermined condition;
make a first determination of, when the content of the reward does not satisfy the predetermined condition, whether the user identification information is associated with terminal identification information on any one of a plurality of wearable terminals belonging to the operator;
transmit, when the content of the reward satisfies the predetermined condition, an input request of the terminal identification information on the wearable terminal of the user to the user terminal;
receive, from the user terminal, the terminal identification information input by the user in response to the request;
make a second determination of whether the terminal identification information received from the user terminal matches the terminal identification information on any one of the plurality of wearable terminals belonging to the operator; and
register the reward information and the user identification information in association with each other based on a result of the first determination or a result of the second determination.

9. A reward granting method to be executed by a user terminal and a server system of an operator that provides a service to a user, the reward granting method comprising:
by the user terminal:
acquiring health data measured by a wearable terminal of the user;
determining, based on the health data, a reward to be granted to the user; and
transmitting, to the server system, reward information indicating a content of the reward and user identification information on the user;
by the server system:
determining, based on the reward information, whether the content of the reward satisfies a predetermined condition;
making a first determination of, when the content of the reward does not satisfy the predetermined condition, whether the user identification information is associated with terminal identification information on any one of a plurality of wearable terminals belonging to the operator; and
transmitting, when the content of the reward satisfies the predetermined condition, an input request of the terminal identification information on the wearable terminal of the user to the user terminal;
by the user terminal, transmitting, to the server system, the terminal identification information input by the user in response to the request; and
by the server system:
making a second determination of whether the terminal identification information transmitted from the user terminal matches the terminal identification information on any one of the plurality of wearable terminals belonging to the operator; and
registering the reward information and the user identification information in association with each other based on a result of the first determination or a result of the second determination.

10. (canceled)
